(19) United States
(12) Patent Application Publication
(10) Pub. No.: US 2014/0127158 A1
(43) Pub. Date: May 8, 2014

(54) COMBINATION THERAPY FOR HEPATITIS C VIRUS INFECTION

(71) Applicant: National Health Research Institute, Miaoli County (TW)

(72) Inventors: Andrew Yueh, New Taipei City (TW); Yu-Sheng Chao, New York, NY (US)

(73) Assignee: National Health Research Institute, Miaoli County (TW)

(21) Appl. No.: 14/073,378

(22) Filed: Nov. 6, 2013

Related U.S. Application Data
(60) Provisional application No. 61/724,127, filed on Nov. 8, 2012.

(51) Int. Cl. A61K 31/427; A61K 31/7056; A61K 31/403; A61K 31/675; A61K 38/21; A61K 31/497

(52) U.S. Cl. CPC A61K 31/427; A61K 38/217; A61K 31/497; A61K 31/403; A61K 31/675; A61K 31/7056
USPC 424/85.5; 514/365; 514/255.05; 514/412; 514/86; 514/43

(57) ABSTRACT

A method of treating hepatitis C virus infection, comprising administering to a subject in need thereof (a) an effective amount of at least one HCV inhibitor selected from the group consisting of an HCV NS3 inhibitor, an HCV NS5B inhibitor, ribavirin, and an IFN-α; and (b) an effective amount of an anti-HCV compound of formula (I).

### CROSS REFERENCE TO RELATED APPLICATION

This application claims priority from U.S. Provisional Application No. 61/724/127 filed on Nov. 8, 2012, both applications being incorporated herein by reference in their entirety.

### BACKGROUND

Hepatitis C virus (HCV) is a small enveloped RNA virus that affects nearly 170 million individuals worldwide, making it a leading cause of hepatitis C and liver disease. HCV infection is responsible for the development of severe chronic liver disease, cirrhosis and associated complications, including liver failure, portal hypertension, and hepatocellular carcinoma.

The main goals of chronic HCV therapy are to eradicate the virus and prevent these potentially life-threatening complications. The mainstays of chronic HCV therapy are PEGylated IFN-α and ribavirin. However, these compounds are poorly tolerated, and may eventually lead to a suboptimal response rate and a high incidence of adverse effects, including is flu-like symptoms, depression and anemia. The chances of sustained viral clearance are only 40-50% for genotype 1 infection, which is the predominant genotype in worldwide populations.

Therefore, the development of specific antiviral therapies for hepatitis C with improved efficacy and better tolerance is a major public health objective.

### SUMMARY

This invention is based on the unexpected discovery that certain anti-HCV compounds, e.g., DBPR110 and DBPR111, when combined with one or more other HCV inhibitors, e.g., telaprevir, boceprevir, sofosbuvir, ribavirin, and interferon-α, exert a synergistic effect on inhibition of HCV.

Accordingly, described herein is a method of treating HCV infection. The method includes administering to a subject in need thereof (a) an effective amount of at least one HCV inhibitor selected from the group consisting of an HCV NS3 inhibitor, an HCV NS5B inhibitor, ribavirin, and an IFN-α; and (b) an effective amount of an anti-HCV compound described below. For example, the anti-HCV compound is DBPR110 or DBPR111.

The details of several embodiments of the invention are set forth in the description below. Other features, objects, and advantages of the invention will be apparent from the description and from the claims.

### DETAILED DESCRIPTION

Described herein is a method of treating HCV infection. The method includes administering to a subject in need thereof a specific combination of two or more compounds that inhibit HCV, e.g., inhibit HCV replication. The combination includes (a) an effective amount of at least one HCV inhibitor selected from the group consisting of an HCV NS3 inhibitor, an HCV NS5B inhibitor, ribavirin, and an IFN-α; and (b) an effective amount of an anti-HCV compound of formula (I):

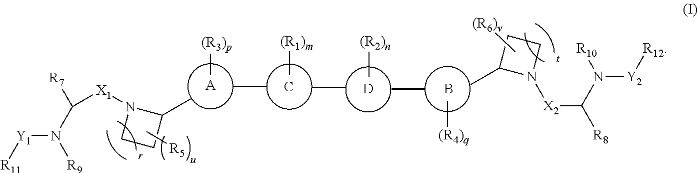

In formula (I), A is

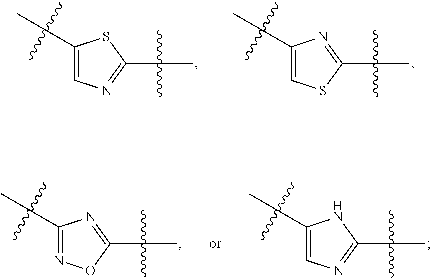 [SMILES: *c1cnc(*)s1 ; *c1csc(*)n1 ; *c1noc(*)n1 ; *c1cnc(*)[nH]1]

### B is

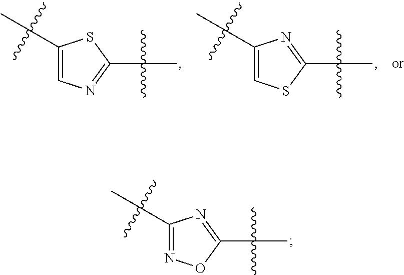 [SMILES: *c1cnc(*)s1 ; *c1csc(*)n1 ; *c1noc(*)n1]

each of C and D, independently, is arylene or heteroarylene; each of R₁, R₂, R₃, R₄, R₅, and R₆, independently, is alkyl, alkenyl, alkynyl, aryl, heteroaryl, cycloalkyl, cycloalkenyl, heterocycloalkyl, halo, heterocycloalkenyl, cyano, or nitro; each of R₇ and R₈, independently, is H, alkyl, alkenyl, alkynyl, aryl, heteroaryl, cycloalkyl, cycloalkenyl, heterocycloalkyl, or heterocycloalkenyl; each of R₉ and R₁₀, independently, is H or alkyl; each of R₁₁ and R₁₂, independently, is H, alkyl, alkenyl, alkynyl, aryl, heteroaryl, cycloalkyl, cycloalkenyl, heterocycloalkyl, or heterocycloalkenyl; each of X₁ and X₂, independently, is C(O) or C(S); each of Y₁ and Y₂, independently, is deleted, SO, SO₂, C(O), C(O)O, C(O)NRₐ, C(S)NRₐ, or SO₂NRₐ, in which Rₐ is H, alkyl, cycloalkyl, heterocycloalkyl, aryl, or heteroaryl; each of m and n, independently, is 0, 1, 2, 3, or 4; each of p and q, independently, is 0 or 1; each of r and t, independently, is 1, 2, or 3; and each of u and v, independently, is 0, 1, 2, 3, 4, 5, 6, 7, or 8.

For example, the anti-HCV compound is of formula (II) below:

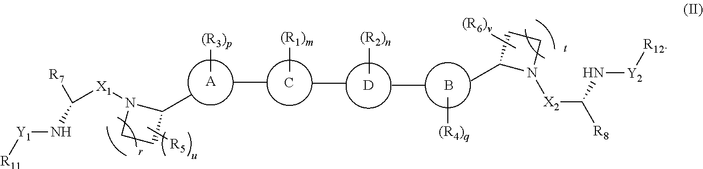

In some embodiments, the anti-HCV compound is of formula (III) below:

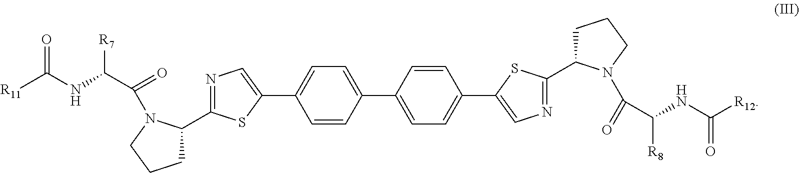

The above-described anti-HCV compounds may include one or more of the following features. Each of A and B is

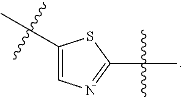 [SMILES: *c1cnc(*)s1]

Each of C and D is phenylene. Each of X₁ and X₂ is C(O). Each of Y₁ and Y₂, independently, is SO₂, C(O), or C(O)O. Each of R₇ and R₈ is phenyl. Each of R₁₁ and R₁₂, independently, is C₁₋₅ alkyl or C₃₋₅ cycloalkyl. Each of t and r is 2. A and B are different. Each of p, m, n, q, u and v is 0. Each of p, m, n, and q is 0, each of u and v is 1, and each R₅ and R₆ is F.

Examples of the above-mentioned anti-HCV compounds are described in U.S. patent application Ser. No. 12/958,734 (published as US2011/0136799).

The term “alkyl” refers to a straight or branched monovalent hydrocarbon containing 1-20 carbon atoms (e.g., C₁-C₁₀). Examples of alkyl include, but are not limited to, methyl, ethyl, n-propyl, i-propyl, n-butyl, i-butyl, and t-butyl. The term “alkenyl” refers to a straight or branched monovalent hydrocarbon containing 2-20 carbon atoms (e.g., C₂-C₁₀) and one or more double bonds. Examples of alkenyl include, but are not limited to, ethenyl, propenyl, and allyl. The term “alkynyl” refers to a straight or branched monovalent hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl.

The term “cycloalkyl” refers to a monovalent saturated hydrocarbon ring system having 3 to 30 carbon atoms (e.g., C₃-C₁₂). Examples of cycloalkyl include, but are not limited to, cyclopropyl, cyclobutyl, cyclopentyl, cyclohexyl, cycloheptyl, and cyclooctyl. The term “cycloalkenyl” refers to a monovalent non-aromatic hydrocarbon ring system having 3 to 30 carbons (e.g., C₃-C₁₂) and one or more double bonds. Examples include cyclopentenyl, cyclohexenyl, and cycloheptenyl. The term “heterocycloalkyl” refers to a monovalent nonaromatic 5-8 membered monocyclic, 8-12 membered bicyclic, or 11-14 membered tricyclic ring system having one or more heteroatoms (such as O, N, S, or Se). Examples of heterocycloalkyl groups include, but are not limited to, piperazinyl, pyrrolidinyl, dioxanyl, morpholinyl, and tetrahydrofuranyl. The term “heterocycloalkenyl” refers to a monovalent nonaromatic 5-8 membered monocyclic, 8-12 membered bicyclic, or 11-14 membered tricyclic ring system having one or more heteroatoms (such as O, N, S, or Se) and one or more double bonds.

The term “aryl” refers to a monovalent 6-carbon monocyclic, 10-carbon bicyclic, or 14-carbon tricyclic aromatic ring system. Examples of aryl groups include, but are not limited to, phenyl, naphthyl, and anthracenyl. The term “arylene” refers to a divalent 6-carbon monocyclic (e.g., phenylene), 10-carbon bicyclic (e.g., naphthylene), or 14-carbon tricyclic aromatic ring system. The term “heteroaryl” refers to a monovalent aromatic 5-8 membered monocyclic, 8-12 membered bicyclic, or 11-14 membered tricyclic ring system having one or more heteroatoms (such as O, N, S, or Se). Examples of heteroaryl groups include pyridyl, furyl, imidazolyl, benzimidazolyl, pyrimidinyl, thienyl, quinolinyl, indolyl, and thiazolyl. The term “heteroarylene” refers to a divalent aromatic 5-8 membered monocyclic, 8-12 membered bicyclic, or 11-14 membered tricyclic ring system having one or more heteroatoms (such as 0, N, S, or Se).

Alkyl, alkenyl, alkynyl, cycloalkyl, heterocycloalkyl, cycloalkenyl, heterocycloalkenyl, aryl, arylene, heteroaryl, and heteroarylene mentioned above include both substituted and unsubstituted moieties. Possible substituents on cycloalkyl, heterocycloalkyl, cycloalkenyl, heterocycloalkenyl, aryl, and heteroaryl include, but are not limited to, C₁-C₁₀ alkyl (e.g., trifluoromethyl), C₂-C₁₀ alkenyl, C₂-C₁₆ alkynyl (e.g., arylalkynyl), C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl (e.g., haloaryl or aryl substituted with halo), aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

The multicyclic compounds described above include the compounds themselves, as well as their salts, their solvates, and their prodrugs, if applicable. A salt, for example, can be formed between an anion and a positively charged group (e.g., amino) on a multicyclic compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on a multicyclic compound. Suitable cations include sodium ion, potassium ion, magnesium ion, calcium ion, and an ammonium cation such as tetramethylammonium ion. The multicyclic compounds also include those salts containing quaternary nitrogen atoms. Examples of prodrugs include esters and other pharmaceutically acceptable derivatives, which, upon administration to a subject, are capable of providing active multicyclic compounds.

Preferably, the anti-HCV compound used in the treatment method is DBPR110, which has the following structure:

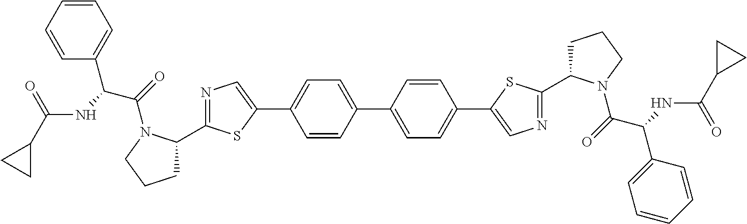 [SMILES: O=C(N[C@@H](C(=O)N1CCC[C@H]1c1ncc(-c2ccc(-c3ccc(-c4cnc([C@@H]5CCCN5C(=O)[C@H](NC(=O)C5CC5)c5ccccc5)s4)cc3)cc2)s1)c1ccccc1)C1CC1]

Another preferred anti-HCV compound is DBPR111, which has the following structure:

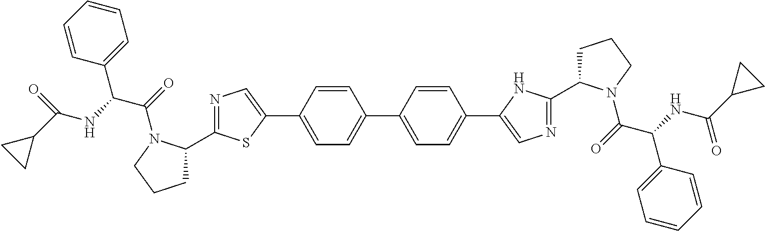 [SMILES: O=C(N[C@@H](C(=O)N1CCC[C@H]1c1ncc(-c2ccc(-c3ccc(-c4cnc([C@@H]5CCCN5C(=O)[C@H](NC(=O)C5CC5)c5ccccc5)s4)cc3)cc2)[nH]1)c1ccccc1)C1CC1]

The above-described anti-HCV compounds can be synthesized using conventional methods or those disclosed in U.S. patent application Ser. No. 12/958,734.

In addition to one of the above-mentioned anti-HCV compounds, one or more (e.g., two) other HCV inhibitors, i.e., an HCV NS3 inhibitor, an HCV NS5B inhibitor, ribavirin, or an IFN-α, are administered to the subject. As examples, a double combination can include (i) an anti-HCV compound and an IFN-α; and (ii) an anti-HCV compound and an HCV NS3 inhibitor. A triple combination can include (i) an anti-HCV compound, an IFN-α, and an HCV NS3 inhibitor; (ii) an anti-HCV compound, an HCV NS3 inhibitor, and an HCV NS5B inhibitor; and (iii) an anti-HCV compound and two different NS5B inhibitors. Various HCV inhibitors are known in is the art. See, e.g., Kwo and Zhao, Clin Liver Dis 15:537-53 (2011); Kwong et al., Curr Opin Pharmacol 8:522-31 (2008); Legrand-Abravanel et al., Expert Opin Investig Drugs 19:963-75 (2010); Liapakis and Jacobson, Clin Liver Dis 15:555-71 (2011); Lemm et al., J Virol 84:482-91 (2010); Naggie et al., J Antimicrob Chemother 65:2063-9 (2010); WO2012/009394; WO2012/018829; and WO2011/046811.

For example, the HCV NS3 inhibitor can be boceprevir or telaprevir (i.e., VX950). An exemplary HCV NS5B inhibitor is sofosbuvir (Pharmasset, Inc., NJ). Ribavirin can inhibit HCV through several mechanisms. As well known in the art, IFN-α, also an anti-HCV agent, can be non-modified or pegylated. These HCV inhibitors can be produced using standard methods or obtained from commercial sources.

To practice the treatment method of this invention, the above-described anti-HCV compound and HCV inhibitor can be administered to a patient together in a single composition, separately at the same time, or at different times. For example, a pharmaceutical composition that contains an effective amount of the anti-HCV compound, an effective amount of the HCV inhibitor, and a pharmaceutically acceptable carrier can be administered to the patient. Alternatively, a pharmaceutical composition containing an anti-HCV compound and a pharmaceutical composition containing another HCV inhibitor can be administered to the patient separately.

As used herein, the term “treating” refers to administering a compound to a subject that has HCV infection, or has a symptom of or a predisposition toward such a disorder, with the purpose to cure, heal, alleviate, relieve, alter, remedy, ameliorate, improve, or affect the above-described disorder, the symptoms of or the predisposition toward it. The term “an effective amount” refers to the amount of the active agent, when used in combination with one or more other active agents, that is required to confer the intended therapeutic effect in the subject.

The above-described anti-HCV compounds and HCV inhibitors can be administered to a subject orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. A compound-containing composition can also be administered in the form of suppositories for rectal administration.

The carrier in the pharmaceutical composition must be “acceptable” in the sense of being compatible with the active ingredient of the formulation (and preferably, capable of stabilizing it) and not deleterious to the subject to be treated. For example, one or more solubilizing agents, which form more soluble complexes with the compounds, or more solubilizing agents, can be utilized as pharmaceutical carriers for delivery of the active compounds. Examples of other carriers include colloidal silicon dioxide, magnesium stearate, sodium lauryl sulfate, and D&C Yellow #10.

The specific example below regarding DBPR110 is to be construed as merely illustrative, and not limitative of the remainder of the disclosure in any way whatsoever. Without further elaboration, it is believed that one skilled in the art can, based on the description herein, utilize the present invention to its fullest extent. All publications cited herein are herein incorporated by reference in their entirety.

### Materials and Methods

(1) E. coli and yeast strains. Frozen, competent E. coli strain C41, derivative of BL21 (DE3) (43), was purchased from OverExpress Inc. Standard yeast medium and transformation methods were used. S. cerevisiae YPH857 was purchased from ATCC. The genotype of YPH857 is MATα ade2-101 lys2-801 ura3-52 trp1-Δ63 HIS5 CAN1 his3-Δ200 leu2-Δ1 cyh2. Competent yeast cells were prepared using the lithium acetate procedure.

(2) Cell culture and HCV inhibitors. Huh-7.5 cells and their derivative HCV replicon cell lines were maintained in Dulbecco's modified Eagle's medium (DMEM, Gibco/BRL) that was supplemented with 100 U/mL penicillin-streptomycin (Gibco/BRL), 0.1 mM nonessential amino acid (NEAA, Gibco/BRL) and 10% fetal bovine serum (FBS) heat inactivated at 37° C. in 5% CO₂. The HCV replicon cell lines were isolated from colonies as described in Lohman et al., Science 285:110-3 (1999). The culture medium for the replicon cells was additionally supplemented with 0.25 to 0.5 mg/mL of G418, unless specified otherwise. Compound DBPR110 and sofosbuvir were synthesized at the Institute of Biotechnology and Pharmaceutical Research at the National Health Research Institutes in Taiwan. Telaprevir (Lin et al., Antimicrob Agents Chemother, 50:1813-22 (2006)) was purchased from Acme Biosciences (Belmont, Calif.). The compounds were stored at −20° C. as 10 to 500 mM dimethyl sulfoxide (DMSO) stock solutions until the assay. IFN-α was purchased from Calbiochem (La Jolla, Calif.) and stored at −80° C.

(3) Inhibitory assay for HCV replicons. Cells were seeded at 1×10⁴ (high-throughput screening assay) or 1×10⁵ (regular assay) cells/well in 96- or 12-well plate, respectively, and incubated for 4 h. The medium was then aspirated and replaced with 0.1 (96-well plate) or 1 (12-well plate) mL of complete medium containing a single compound or combinations of compounds in serial concentration(s). The plates with compounds were incubated for 72 h and then assayed for luciferase expression (Promega). The EC₅₀ of each compound was determined independently and used to determine the range of concentrations used for the combination experiments. All data are presented as the means±standard deviations (SD) from three independent experiments. The selectivity index (SI) was calculated as the ratio of the CC₅₀ to the EC₅₀.

(4) Cytotoxicity assay. The sensitivity of the cell lines to inhibitors was examined using a 3-(4,5-Dimethylthiazol-2-yl)-2,5-diphenyltetrazolium bromide (MTT) assay. Briefly, Huh-7.5 cells were plated at a density of 1×10⁵ cells per well in 12-well plates containing 1 mL of culture medium for 4 h. Serial dilutions of the compounds or DMSO (negative control) were added, and the plates were incubated for an additional 72 h. The MTT reagent was then added to each well, and the plates were incubated for 3 h at 37° C. in a humidified 5% CO₂ atmosphere before reading at a wavelength of 563 nm using an ELISA plate reader. All data are presented as the means +/−SD from four independent experiments.

(5) Small molecule inhibition of HCV infectivity. To quantify the inhibitory effect of DBPR110 on HCV particle formation, HCV replication in DBPR110-treated and untreated cells was quantified using a luciferase activity assay, as described previously. See, e.g., Wakita et al., Nat Med 11:791-6 (2005); and Zhang et al., Antimicrob Agents Chemother 52:666-74 (2008). In vitro-transcribed RNA derived from full-length HCV2a JFH1 infectious cDNA clone with the luciferase reporter gene was delivered to Huh-7.5 cells by electroporation. The cells were seeded at 1×10⁵ cells per well in 12-well plates and incubated for 4 h. The medium was then aspirated and replaced with 1 mL of complete medium containing DBPR110 in serial concentration. The plates with compounds were incubated for 72 h and the medium were then used to infect Huh-7.5 cells. Huh-7.5 cells were seeded in 12-well plates (1×10⁵ cells/well) in DMEM with 10% FBS for 24 h before infection. The HCV cell culture (HCVcc)-containing supernatant per well was added to the Huh-7.5 cells. After 72 h of incubation at 37° C., the total cell lysate was assayed for luciferase expression (Promega).

(6) Isolation of resistant replicons. Selection of resistant replicon cells was performed by growing HCV genotype 1b Con1 and 2a JFH1 replicon cells in medium containing 0.2 or 200 nM and 60 nM or 1 μM of DBPR110, respectively. Medium containing the compound was added to monolayers of HCV1b-neo replicon cells at ˜25% confluence in the presence of 0.2 to 0.4 mg/mL of G418. Replicon cells maintained in the presence of dimethyl sulfoxide (DMSO) were used as a control. After 40 days, total RNA was isolated from both the control replicon cells and homogeneous cell lines containing compound using the TRIzol reagent (Invitrogen, Carlsbad, Calif.) according to the manufacturer's protocol. The RNA was amplified by reverse transcription-PCR (RT-PCR). The PCR products of NS3-NS5B were gel-purified and subcloned into the pRS-Luc-HCV1bRep vector to replace the parental NS3-NS5B by homologous recombination in yeast. Thirty-six colonies of plasmids were purified from the yeast cells and re-amplified in the E. coli strain C41 strain for DNA sequencing.

(7) Construction of molecular clones containing resistance mutations. To create point mutations derived from the resistant clones, the amino-acid substitutions P58S, P58T, P58L, Y93H, Y93N, Y93C, V153M, M202L, and M265V were introduced into the phRlu-HCV1b plasmid, and T24A, P58L, Y93N and Y93H were introduced into the HCV2a plasmid, either individually or in combination using PCR. The PCR products were gel-purified and joined by overlapping PCR to form the fragments containing the following single, double or triple mutations for homologous recombination with linearized phRlu-HCV1b plasmids (digested with is HpaI): V153M+M202L+M265V, Y93N+V153M+M202L+M265V, and Y93H+V153M+M202L+M265V. The mutant replicon plasmids were purified from yeast cells and then re-amplified and maintained in the E. coli strain C41 strain. All constructs were sequenced to confirm the presence of the desired mutations and to ensure that there were no additional changes.

(8) RNA transcription and transient replicon assay. The RNA transcripts were synthesized in vitro using ScaI-digested DNAs and the T7 MegaScript transcription kit (Ambion) according to the manufacturer's directions. A transient replicon assay was performed to quantify the compound-mediated inhibition of viral translation (Dears et al., J Virol 79:4599-609 (2005)). RNA transcripts were transfected into Huh-7.5 cells by electroporation, as described previously. See, e.g., Blight et al., J Virol 76:13001-14 (2002). A specific concentration of DBPR110 or the control medium was added to each well, and the cells were assayed to determine the luciferase activity at 4 h and 72 h post-transfection. The cells were lysed for luminometry and the luciferase assay was performed by mixing 5 μl of lysate with 25 μl of the Renilla Luciferase Assay Reagent (Promega). For quantification of the compound-mediated inhibition, the relative luciferase activity derived from the mock-treated cells was set to 100% (Zou et al., Virology 384:242-52 (2009)).

(9) Serum shift assay. In the serum shift assay, the inhibitory activity of DBPR110 was determined using replicon 1b in the presence of 10, 20, 30, 40 or 50% fetal bovine serum, or 10 or 40% of extracellular normal human serum. In the absence or presence of serial dilution of DBPR110, the percentage of inhibition was determined by a 50% or 90% reduction in Renilla luciferase activity (EC₅₀ or EC₉₀, respectively) compared to the control after 72 h incubation.

(10) Energy calculation. The docking module implemented in the program Insight II from Accelrys Inc. (San Diego, Calif.) was used to calculate the binding energy between DBPR110 and the HCV NS5A variants. The hydrogen atoms were first added to the compounds and protein. The potentials for the DBPR110 and HCV NS5A variants were subsequently assigned by using the Consistent Force Field (CFF). The parameters for the assignment of potentials using the CFF force field were set at the default values. The interaction energy, a combination of the van der Waals energy and electrostatic energy, between the DBPR110 and HCV NS5A variants was finally calculated using the docking module in the Insight II program.

(11) Computational modeling. The Discovery Studio 2.1 program from Accelrys Inc. (San Diego, Calif.) was used to build the computational models of the HCV NS5A protein. The three-dimensional structure of the parental HCV NS5A was used as a template to perform energy minimization. The force fields of the conformations were further verified using Chemistry at HARvard Macromolecular Mechanics (CHARMm), and the parameters used were set at the default values.

(12) Statistical analysis. The reported values are the average of three independent measurements and expressed as mean±standard deviation. The statistical significance of the difference between the means of the experimental groups was tested by the Student t test for unpaired data. A difference was considered statistically significant when P value was <0.05 (Sigma Plot 10 software, Systat Software, San Jose, Calif.).

(13) Inhibitor combination study. Luciferase reporter-linked HCV replication assays were used to evaluate the potential use of DBPR110 in combination with IFN-α, ribavirin, NS3 protease inhibitors (telaprevir and boceprevir) and a nucleotide inhibitor of NS5B (sofosbuvir). For the combination index model, the cells were incubated for 72 h with serial dilutions of IFN-α, ribavirin, telaprevir, boceprevir, or sofosbuvir, and DBPR110 below their cytotoxic concentrations. CalcuSyn (Biosoft) was used to analyze the data obtained from the 72-h luciferase-based HCV replicon assay and quantify the differences between the observed effects and predicted ones. Compound interactions and concentration ratios were quantified using the approach described by Chou and Talalay. The degrees of synergistic and additive effects were evaluated using the median-effect principle with the combination index (CI) calculation. The combination indices (CIs) at the EC₅₀, EC₇₀, and EC₉₀ were also determined. In total, six combinations were evaluated with three to eight experiment replicates per condition. By convention, a CI of 0.9 was considered synergistic, a CI of >0.9 or <1.1 was considered additive, and a CI of >1.1 was deemed antagonististic.

### Identification of DBPR110 as a Potent Inhibitor of HCV Replication

DBPR110, a novel di-thiazole analogue, was identified as an inhibitor of HCV replication, having an EC₅₀ value in the picomolar range for the HCV1b and 2a replicon cell lines. DBPR110 displayed improved potency against the genotype 1b and 2a replicons, as well as the 2a infectious virus, all with calculated CC₅₀ values of over 50 μM and EC₅₀ values of 3.9, 228.8, and 18.3 pM, respectively, as assessed by luciferase reporter activity. See Table 1 below. DBPR110 displayed an in vitro selective index (CC₅₀/EC₅₀) of over 12,800,000 for the HCV genotype 1b replicon, 173,130 for the genotype 2a replicon, and 720,461 for the 2a infectious virus. Moreover, the susceptibility of genotype 1b to DBPR110 was 74-fold greater than that of genotype 2a replicon cells. Another di-imidazole analogue HCV inhibitor, BMS-790052, was shown to have comparable potency against HCV1b (EC₅₀=9 pM) and 2a replicon activity (EC₅₀=71 pM) (Gao et al., Nature 465:96-100 (2010)). Analysis of the potency of DBPR110 by real-time PCR revealed similar effects.

To distinguish inhibition of viral translation from inhibition of RNA synthesis, the reduction rate of reporter gene expression level was monitored as an indicator of the inhibitory activity of DBPR110. The HCV1b reporter replicon construct, pRS-Luc-HCV1bRep, was transcribed in vitro and transfected into Huh7.5 cells. The luciferase activity was monitored several times over a period of 72 hours posttransfection. The level of luciferase activity was sustained until 72 hours posttransfection in the absence of DBPR110. The luciferase activity peaked within the first 8 hours posttransfection and also after 72 hours posttransfection, representing viral translation and RNA replication, respectively. The luciferase activity was measured at 4, 8, 24, 48, and 72 hours posttransfection. DBPR110 had a minimal effect on the Rluc signals at 4 and 8 hours posttransfection, but the signals were significantly reduced at 24, 48, and 72 hours posttransfection, respectively (P<0.001). In summary, the data demonstrated that DBPR110 significantly suppressed viral RNA synthesis.

TABLE 1
Potency of DBPR110 on HCV replicon cell
line and virus particle formation

| | Luciferase activity assay | | CC₅₀^{a} | Selective |
|---|---|---|---|---|
| HCV Genotype | EC₅₀^{a} (pM) | EC₉₀^{a} (pM) | (μM) | index |
| Genotype 1b, Con1 | 3.9 ± 0.9 | 8.2 ± 1.8 | >50 | >12,800,000 |
| Genotype 2a, JFH1 | 228.8 ± 98.4 | 464.7 ± 96.6 | >50 | >173,130 |
| Infectious HCV, | 18.3 ± 2.6 | 257.5 ± 50.2 | >50 | >720,461 |
| Genotype 2a, JFH1 | | | | |

^{a}Means ± standard deviations determined from the parental cell line (n ≧ 3).

### Isolation and Characterization of Genotype 1b Replicons Resistant to DBPR110

To characterize the resistance profile of DBPR110, cell clones resistant to DBPR110 were obtained by culturing HCV genotype 1b replicon cells in the presence of G418 and increasing concentrations of DBPR110 ranging from 50- to 50,000-fold the EC₅₀ value. The selection experiment revealed that replication of the cognate replicons was resistant to inhibition by DBPR110 and that they displayed a loss of potency as compared to the parental cell lines. Compared to the parental cells, which had an EC₅₀ value of 0.0039 nM, the DBPR110-resistant cells (i.e., DBPR110R) were greater than 14,000-fold more resistant, having an EC₅₀ value of more than 55 nM.

Direct DNA sequencing of individual clones containing NS3-NS5B from 1b-resistant cells revealed multiple changes in the N-terminus of NS5A (summarized in Table 3 below). P58L/T (20%), Y93N/H (73%), V153M (53%), M202L (47%), and M265V (40%) were the predominant mutations observed in 0.2 nM DBPR110-resistant clone selections. See Table 2 below. In total, 100% of the cDNA clones isolated from the cells treated with 200 nM DBPR110 contained the mutations Y93N, V153M, M202L, and M265V. Again, see Table 2 below. None of these amino acid substitutions was observed in the NS5A cDNA clones isolated from the DMSO-treated control cells. Substitutions at P58 and Y93 of NS5A are common mutations in HCV drug resistance studies, signifying that these residues play an important role in the drug-resistant functions of HCV. Other frequent mutations were checked in the 5′ UTR, 3′ UTR and the other non-structural regions of DBPR110-resistant HCV replicon cells. No such mutations were found outside of NS5A region.

TABLE 2
Amino acid changes in genotype 1b HCV NS5A derived from cells resistant to 0.2 or 200 nM DBPR110

| | Amino acid | DBPR110 resistant individual clone^{a} | | | | | | | | | | | | | | |
|---|---|---|---|---|---|---|---|---|---|---|---|---|---|---|---|---|
| DBPR110 | pB77 | p1 | p18 | p6 | p9 | p15 | p2 | p19 | p14 | p21 | p7 | p10 | p16 | p20 | p17 | p22 |
| 0.2 nM | 58 P | L | L | | | | | | | T | | | | | | |
| | 93 Y | | N | N | N | N | N | | | | H | H | H | H | H | H |
| | 153 V | | M | M | M | M | M | M | M | M | | | | | | |
| | 202 M | | L | L | L | L | L | L | L | | | | | | | |
| | 265 M | | V | V | | V | V | V | V | | | | | | I | |
| | Amino acid | DBPR110 resistant individual clone^{a} | | | | | | | | | | | | | | |
| | pB77 | p1 | p2 | p3 | p4 | p5 | p6 | p7 | p8 | p9 | p10 | p11 | | | | |
| 200 nM | 93 Y | N | N | N | N | N | N | N | N | N | N | N | | | | |
| | 153 V | M | M | M | M | M | M | M | M | M | M | M | | | | |
| | 202 M | L | L | L | L | L | L | L | L | L | L | L | | | | |
| | 265 M | V | V | V | V | V | V | V | V | V | V | V | | | | |

^{a}p stands for the plasmid derived from DBPR110 resistant individual clones.

### Validation of the Genotype 1b Mutations Responsible for the Resistant Phenotype.

To determine the contributions of specific mutations to inhibitor sensitivity, the resistant phenotypes were further validated by engineering mutations into a HCV genotype 1b replicon that contained a luciferase reporter gene, which can be used to monitor replication in a transient reporter assay. The replication of the parental and mutant clone replicons was monitored over time in the presence or absence of DBPR110. Maximum replication efficiency for both the parental and mutant RNAs was determined to be 72 h post-transfection.

As shown in Table 3 below, the replication efficiencies of the P58S, P58T, P58L, Y93N, Y93H, and Y93C replicons were 42±10, 40±15, 19±8, 8±3, 8±4, and 9±6% of the level of the parental replicon at 72 h, respectively. This result indicates that these resistant mutants had reduced fitness, with the amino acid substitutions Y93N/H/C showing the lowest replication capacity. Again see Table 3 below. It was shown previously that substitutions at residue 93 also had a great impact on replication fitness. See, Fridell et al., Antimicrob Agents Chemother 54:3641-50. The replication efficiencies of V153M, M202L, and M265V were 70±17, 106±37, and 87±23% of the level of the parental replicon, respectively, indicating that the V153M, M202L, and M265V mutations did not affect fitness. See Table 3 below. Our data revealed that most of the DBPR110-resistant clones contained a combination of two or four amino acid substitutions at residues 58, 93, 153, 202, or 265. See Table 2 above.

The complexity of the resistance pattern was verified by the analysis of individual cDNA clones. All of the 200 nM DBPR110-resistant clones contained the combination Y93N+V153M+M202L+M265V. See Table 2 above. Furthermore, to determine the phenotypes of the variants with linked mutations, replicons with the following representative combinations were tested in transient replication assays: V153M+M202L+M265V, Y93N+V153M+M202L+M265V, and Y93H+V153M+M202L+M265V. The Y93N+V153M+M202L+M265V and Y93H+V153M+M202L+M265V variants exhibited an impaired replication capacity of 16-32% relative to the parental clone. See Table 3 below.

The individual amino acid substitutions P58S/T/L and Y93N/H/C exhibited different levels of resistance to DBPR110, with increasing EC₅₀ values ranging from 25- to 2,547-fold above the parental control. See Table 3 below. When Y93N was combined with V153M, M202L, and M265V on the same replicon, the effects on the inhibitor increased dramatically to give a 2,547-fold boost in resistance. On the other hand, V153M, M202L, and M265V identified in a single NS5A cDNA clone did not affect DBPR110 potency as a single mutation, but the combination of Y93N+V153M+M202L+M265V or Y93H+V153M+M202L+M265V produced a 18,217- or 5,824-fold resistance, respectively. Again, see Table 3 below. This suggests that the primary conformation of NS5A, or of NS5A in the replication complex, is the predominant determinant for inhibitor sensitivity, while residues 58, 93, 153, 202, and 265 are the determinants for resistance selection in genotype 1b of HCV.

TABLE 3
Effects of genotype 1b HCV NS5A amino acid substitutions on DBPR110 potency

| Amino acid | Replication | | Fold | | Fold |
|---|---|---|---|---|---|
| substitution (s) | leve1^{a} | EC₅₀^{a} (pM) | resistance | EC₉₀^{a} (pM) | resistance |
| Parental | 100 | 1.5 ± 0.6 | 1 | 4.2 ± 2.1 | 1 |
| P58S | 42 ± 10 | 38 ± 14 | 25 | 64 ± 11 | 15 |
| P58T | 40 ± 15 | 243 ± 40 | 162 | 1303 ± 219 | 310 |
| P58L | 19 ± 8 | 564 ± 194 | 376 | 2731 ± 909 | 650 |
| Y93N | 8 ± 3 | 3,821 ± 1,677 | 2,547 | 13,305 ± 3,416 | 3,168 |
| Y93H | 8 ± 4 | 1,408 ± 293 | 939 | 7,337 ± 2,206 | 1,747 |
| Y93C | 9 ± 6 | 78 ± 40 | 52 | 177 ± 62 | 42 |
| V153M | 70 ± 17 | 1.3 ± 0.5 | 1 | 4.1 ± 1.9 | 1 |
| M202L | 106 ± 37 | 2.1 ± 0.6 | 1 | 5.0 ± 1.4 | 1 |
| M265V | 87 ± 23 | 2.0 ± 0.9 | 1 | 5.1 ± 1.7 | 1 |
| V153M + M202L + | 157 ± 52 | 1.1 ± 0.5 | 1 | 3.1 ± 1.1 | 1 |
| M265V | | | | | |
| Y93N + V153M + | 16 ± 4 | 27,326 ± 12,349 | 18,217 | 98,912 ± 30,548 | 23,550 |
| M202L + M265V | | | | | |
| Y93H + V153M + | 32 ± 10 | 8,736 ± 2,370 | 5,824 | 37,710 ± 6,970 | 8,979 |
| M202L + M265V | | | | | |

^{a}Means ± standard deviations determined from transient transfection assays (n ≧ 3).

### Isolation and Characterization of Genotype 2a Replicons Resistant to DBPR110

Cell clones resistant to DBPR110 were obtained by culturing HCV genotype 2a replicon cells in the presence of G418 and increasing concentrations of DBPR110 ranging from 60 to 1000 nM. The selection experiment revealed that the replication of the cognate replicons was resistant to inhibition by DBPR110 and that they displayed a loss of potency compared to the parental cell lines. Direct DNA sequencing of the individual clones containing NS3-NS5B from 2a-resistant cells revealed multiple changes in the N-terminus of NS5A, as summarized in Table 4 below. More specifically, the predominant mutations observed in the 60 nM DBPR110-resistant clone selections were T24A (50%) and P58L (50%). In total, 100% of the cDNA clones isolated from the cells treated with 1 μM DBPR110 contained only the mutation Y93H. None of these amino acid substitutions were detected in the NS5A cDNA clones isolated from the DMSO-treated control cells.

TABLE 4
Amino acid changes in genotype 2a HCV NS5A derived
from cells resistant to 60 nM or 1 μM DBPR110

| | Amino acid | DBPR110 resistant individual clone^{a} | | | | | | | | |
|---|---|---|---|---|---|---|---|---|---|---|
| DBPR110 | pB77 | p1 | p2 | p3 | p4 | p9 | p6 | p5 | p8 | |
| 60 nM | 24 T | A | A | | | | | A | A | |
| | 58 P | | | L | L | L | L | | | |
| | Amino acid | DBPR110 resistant individual clone^{a} | | | | | | | | |
| | pB77 | p1 | p2 | p3 | p4 | p5 | p6 | p7 | p8 | p9 |
| 1 μM | 93 Y | H | H | H | H | H | H | H | H | H |

^{a}p stands for the plasmid derived from DBPR110 resistant individual clones.

### Validation of Genotype 2a Mutations Responsible for the Resistant Phenotype.

When tested in replicon transient assays, the T24A, P58L and Y93N/H mutations reduced susceptibility to DBPR110. As shown in Table 5 below, the replication efficiencies of the T24A, P58L, Y93N, and Y93H replicons were 120±12, 154±20, 103±28, and 192±13% of the parental replicon at 72 h, respectively. These results showed that these resistant mutants did not have impaired fitness. The individual amino acid substitutions T24A, P58L, Y93N, and Y93H exhibited different levels of resistance to DBPR110 with increasing EC₅₀ values ranging from 65- to 3,041-fold above the parental control. Again see Table 5 below. The substitution of Y93H had the greatest impact on susceptibility to DBPR110. It indicated that the primary conformation of NS5A is the predominant determinant for inhibitor sensitivity in genotype 2a, while residues 24, 58, and 93 are the determinants for resistance selection in genotype 2a of HCV.

TABLE 5
Effects of genotype 2a HCV NS5A amino acid substitutions on DBPR110 potency

| Amino acid | | Fold | | Fold | Replication |
|---|---|---|---|---|---|
| substitution | EC₅₀^{a} (pM) | resistance | EC₉₀^{a} (pM) | resistance | level^{a} |
| Parental | 250 ± 32 | 1 | 592 ± 70 | 1 | 100 |
| T24A | 16,245 ± 4,547 | 65 | 63,488 ± 8,467 | 107 | 120 ± 12 |
| P58L | 52,953 ± 8,045 | 212 | 89,348 ± 27,926 | 151 | 154 ± 20 |
| Y93N | 51,766 ± 6,307 | 207 | 85,243 ± 15,920 | 144 | 103 ± 28 |
| Y93H | 760,167 ± 175 | 3,041 | >5,000,000 | >8,446 | 192 ± 13 |

^{a}Means ± standard deviations determined from transient transfection assays (n ≧ 3).

### Protein Binding Activity of DBPR110

To evaluate the effect of serum protein binding on DBPR110 activity, fetal bovine serum (FBS) and normal human serum (NHS) were used. Our results revealed that, in the presence of 10, 20, 30, 40, and 50% FBS, the EC₅₀ values were 4.3±0.8, 8.1±1.6, 7.9±0.9, 13.2±1.7, and 21.5±10 pM, respectively, and the EC₉₀ values were 9.3±3.4, 23.8±11, 21.6±17, 35.1±7.4, and 41.9±7.2 pM, respectively. In the presence of 10 and 40% NHS, the EC₅₀ values were 33.5±0.4 and 210.9±6.3 pM, respectively, and the EC₉₀ values were 41.6±1.3 and 588.1±45.9 pM, respectively. See Table 6 below. While the activity of DBPR110 at higher serum concentrations was more favorable than that at lower levels, the EC₅₀ and EC₉₀ values were increased 1.9- to 6.3-fold and 2.6- to 14.1-fold, respectively. Again, see Table 6 below. These results indicated that there is an apparent minor shift in the potency of DBPR110 in the presence of higher serum concentrations.

TABLE 6
Effects of serum on the antiviral activity
of DBPR110 in HCV1b replicon cell lines

| | HCV1b replicon results | | | |
|---|---|---|---|---|
| Serum^{b} (%) | E₅₀^{a} (pM) | Shift fold | EC₉₀^{a} (pM) | Shift fold |
| FBS | | | | |
| 10 | 4.3 ± 0.8 | 1.0 | 9.3 ± 3.4 | 1.0 |
| 20 | 8.1 ± 1.6 | 1.9 | 23.8 ± 11.0 | 2.6 |
| 30 | 7.9 ± 0.9 | 1.8 | 21.6 ± 17.0 | 2.3 |
| 40 | 13.2 ± 1.7 | 3.1 | 35.1 ± 7.4 | 3.8 |
| 50 | 21.5 ± 10.0 | 5.0 | 41.9 ± 7.2 | 4.5 |
| NHS | | | | |
| 10 | 33.5 ± 0.4 | 1.0 | 41.6 ± 1.3 | 1.0 |
| 40 | 210.9 ± 6.3 | 6.3 | 588.1 ± 45.9 | 14.1 |

^{a}Means ± standard deviations determined from the parental cell line (n = 3).
^{b}FBS, fetal bovin serum; NHS, normal human serum.

### Structural Studies

HCV NS5A mutations can be associated with either altered drug-binding efficiency or drug resistance. Here, computational modeling was employed to give structural insights. The three-dimensional HCV NS5A structure (Love et al., J Virol 83:4395-403 (2009)) and the Discovery Studio 2.1 program (Accelrys, Inc) were applied to build a model by mutating residues and performing energy minimization. See Table 7 below. The DBPR110-associated mutation points, P58 and Y93 were mapped onto a HCV NS5A crystal structure of the DBPR110-NS5A protein complex. The results of modeling suggest that DBPR110 binds directly to the dimer interface of HCV NS5A.

The binding energy of DBPR110 in the HCV NS5A variants was calculated as a whole to gain a better insight into the role played by the DBPR110-resistant variants in the interactions with DBPR110. See Table 7 below. Parental NS5A and NS5A accompanied by V153M showed the most stable conformation with DBPR110, with −26.79 and −29.06 kcal mol⁻¹ of binding energy (van der Waals energy and electrostatic energy), respectively, followed by P58L with −4.38 kcal mol⁻¹ and Y93H, with 18.63 kcal mol⁻¹ and Y93N showed the least stability, with 79.30 kcal mol⁻¹ of binding energy. Again, see Table 7 below. Thus, mutation of these residues seems to affect affinity for DBPR110.

TABLE 7
EC₅₀ of DBPR110 resistant variants and
binding energy of DBPR110 to HCV NS5A

| Amino acid substitution | | Parental | V153M | P58L | Y93H | Y93N |
|---|---|---|---|---|---|---|
| EC₅₀ (DBPR110, pM) | | 1.5 | 1.3 | 564 | 1408 | 3821 |
| Binding | VdW + Elect | −26.79 | −29.06 | −4.38 | 18.63 | 79.30 |
| Energy | (kcal/mol) | | | | | |
| | VdW | −23.63 | −35.16 | −11.08 | 21.14 | 87.63 |
| | Contribution | | | | | |
| | (kcal/mol) | | | | | |
| | Elect | −3.16 | 6.10 | 6.70 | −2.51 | −8.33 |
| | Contribution | | | | | |
| | (kcal/mol) | | | | | |

Combination Therapy of DBPR110 with Other HCV Inhibitors

Standard care or single-agent therapies for viral infections often lead to production of quasi-species, which increases the possibility of clinical drug resistance. Therefore, more effective and better-tolerated combination therapies to decrease the emergence of viral resistance are greatly needed.

In order to evaluate the effect of DBPR110 used in combination with other HCV inhibitors, the inhibitory activity of pair-wise combinations of IFN-α, ribavirin, telaprevir, boceprevir, or sofosbuvir with DBPR110 were analyzed using a genotype 1b replicon encoding a luciferase reporter gene. In this system, DBPR110 had a calculated EC₅₀ value of 3.3±0.8 pM, whereas IFN-α, ribavirin, telaprevir, boceprevir, and sofosbuvir had respective EC₅₀ values of 35.1±4.7 IU/mL, 20.5±3.5 μM, 301.6±2.8 nM, 360.6±19.9 nM, and 91.5±18.3 nM. See Table 8 below.

TABLE 8
Potency of DBPR110, IFN-α, ribavirin, telaprevir,
boceprevir, and sofosbuvir on HCV-1b replicon cell lines

| Compound | EC₅₀^{a} | EC₉₀^{a} | CC₅₀^{a} |
|---|---|---|---|
| DBPR110 (pM) | 3.3 ± 0.8 | 7.4 ± 0.8 | >50,000 |
| IFN-α (IU/mL) | 35.1 ± 4.7 | 327.0 ± 0.01 | >2,000 |
| Ribavirin (μM) | 20.5 ± 3.5 | 95.0 ± 20.1 | >200 |
| Telaprevir (nM) | 301.6 ± 2.8 | 911.9 ± 75.4 | >5,000 |
| Boceprevir (nM) | 360.6 ± 19.9 | 962.0 ± 21.5 | >5,000 |
| Sofosbuvir (nM) | 91.5 ± 18.3 | 323.0 ± 66.1 | >5,000 |

^{a}Means ± standard deviations determined from the HCV1b replicon cells (n ≧ 3).

DBPR110 was mixed with IFN-α, ribavirin, telaprevir, boceprevir, or sofosbuvir at different ratios and serial dilutions of each mixture were generated thereafter. The degree of inhibition for each drug combination was analyzed according to the median effect principle using the combination index calculation at 50%, 75%, and 90%. In three independent experiments, the combination of DBPR110 with IFN-α, ribavirin, telaprevir, boceprevir, or sofosbuvir produced synergistic effects at the 50%, 75%, and 90% effective doses. See Table 9 below. No cytotoxicity was observed for DBPR110, IFN-α, ribavirin, telaprevir, boceprevir, or sofosbuvir at the concentrations used in these experiments.

TABLE 9
Synergistic effects of DBPR110 in combination with IFN-α, ribavirin,
telaprevir, boceprevir, or sofosbuvir at 50%, 75%, and 90% effective doses

| Combination | Ratio, DBPR110 | CI value for^{a}: | | | |
|---|---|---|---|---|---|
| compound | to other compound | ED₅₀ | ED₇₅ | ED₉₀ | Influence |
| IFN-α | 1:1 | 0.50 ± 0.17 | 0.54 ± 0.19 | 0.58 ± 0.20 | Synergistic |
| | 2.5:1 | 0.57 ± 0.31 | 0.59 ± 0.31 | 0.61 ± 0.33 | Synergistic |
| | 1:2.5 | 0.45 ± 0.08 | 0.49 ± 0.09 | 0.54 ± 0.12 | Synergistic |
| Ribavirin | 1:1 | 0.75 ± 0.08 | 0.68 ± 0.03 | 0.62 ± 0.02 | Synergistic |
| | 2.5:1 | 0.71 ± 0.28 | 0.70 ± 0.19 | 0.69 ± 0.10 | Synergistic |
| | 1:2.5 | 0.52 ± 0.19 | 0.49 ± 0.11 | 0.47 ± 0.04 | Synergistic |
| Telaprevir | 1:1 | 0.43 ± 0.27 | 0.42 ± 0.18 | 0.43 ± 0.10 | Synergistic |
| | 2.5:1 | 0.67 ± 0.42 | 0.63 ± 0.33 | 0.60 ± 0.23 | Synergistic |
| | 1:2.5 | 0.34 ± 0.16 | 0.34 ± 0.11 | 0.34 ± 0.07 | Synergistic |
| Boceprevir | 1:1 | 0.46 ± 0.22 | 0.38 ± 0.19 | 0.31 ± 0.17 | Synergistic |
| | 2.5:1 | 0.29 ± 0.14 | 0.29 ± 0.15 | 0.29 ± 0.16 | Synergistic |
| | 1:2.5 | 0.47 ± 0.25 | 0.43 ± 0.26 | 0.39 ± 0.28 | Synergistic |
| Sofosbuvir | 1:1 | 0.62 ± 0.11 | 0.56 ± 0.10 | 0.51 ± 0.08 | Synergistic |
| | 2.5:1 | 0.77 ± 0.17 | 0.70 ± 0.12 | 0.64 ± 0.08 | Synergistic |
| | 1:2.5 | 0.48 ± 0.07 | 0.42 ± 0.04 | 0.38 ± 0.01 | Synergistic |

^{a}Means ± standard deviations determined from the HCV1b replicon cells (n ≧ 3).

DBPR110 was also tested in triple drug combinations with IFN-α, and ribavirin, telaprevir, boceprevir, or sofosbuvir using genotype 1b replicon cells, as summarized in Table 10. Synergistic effects were observed at 50%, 75%, and 90% effective doses using the triple combinations. See Table 10 below.

TABLE 10
Synergistic effects of DBPR110 and IFN-α in
combination with ribavirin, telaprevir, boceprevir,
or sofosbuvir at 50%, 75%, and 90% effective doses

| | CI value for^{a}: | | | |
|---|---|---|---|---|
| Ratio (1:1:1) | ED₅₀ | ED₇₅ | ED₉₀ | Influence |
| DBPR110 + | 0.36 ± 0.05 | 0.3 ± 0.02 | 0.25 ± 0.004 | Synergistic |
| IFN-α + | | | | |
| Ribavirin | | | | |
| DBPR110 + | 0.40 | 0.35 | 0.31 | Synergistic |
| IFN-α + | | | | |
| Telaprevir | | | | |
| DBPR110 + | 0.41 ± 0.12 | 0.37 ± 0.10 | 0.34 ± 0.10 | Synergistic |
| IFN-α + | | | | |
| Boceprevir | | | | |
| DBPR110 + | 0.19 ± 0.09 | 0.18 ± 0.09 | 0.17 ± 0.09 | Strong |
| IFN-α + | | | | Synergistic |
| Sofosbuvir | | | | |

^{a}Means ± standard deviations determined from the HCV1b replicon cells (n ≧ 3).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of treating hepatitis C virus infection, comprising administering to a subject in need thereof (a) an effective amount of at least one HCV inhibitor selected from the group consisting of an HCV NS3 inhibitor, an HCV NS5B inhibitor, ribavirin, and an IFN-α; and (b) an effective amount of an anti-HCV compound of formula (I): 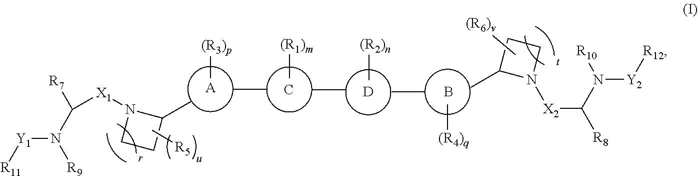
wherein
A is 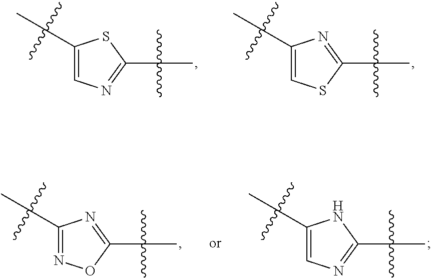 [SMILES: *c1cnc(*)s1 ; *c1csc(*)n1 ; *c1noc(*)n1 ; *c1cnc(*)[nH]1]
B is 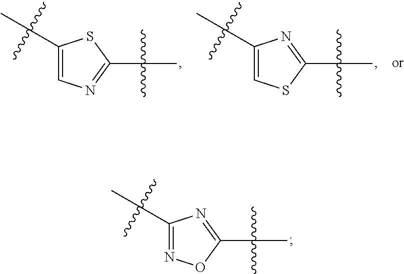 [SMILES: *c1cnc(*)s1 ; *c1csc(*)n1 ; *c1noc(*)n1]
each of C and D, independently, is arylene or heteroarylene;
each of R₁, R₂, R₃, R₄, R₅, and R₆, independently, is alkyl, alkenyl, alkynyl, aryl, heteroaryl, cycloalkyl, cycloalkenyl, heterocycloalkyl, halo, heterocycloalkenyl, cyano, or nitro;
each of R₇ and R₈, independently, is H, alkyl, alkenyl, alkynyl, aryl, heteroaryl, cycloalkyl, cycloalkenyl, heterocycloalkyl, or heterocycloalkenyl;
each of R₉ and R₁₀, independently, is H or alkyl;
each of R₁₁ and R₁₂, independently, is H, alkyl, alkenyl, alkynyl, aryl, heteroaryl, cycloalkyl, cycloalkenyl, heterocycloalkyl, or heterocycloalkenyl;
each of X₁ and X₂, independently, is C(O) or C(S);
each of Y₁ and Y₂, independently, is deleted, SO, SO₂, C(O), C(O)O, C(O)NRₐ, C(S)NRₐ, or SO₂NRₐ, in which Rₐ is H, alkyl, cycloalkyl, heterocycloalkyl, aryl, or heteroaryl;
each of m and n, independently, is 0, 1, 2, 3, or 4;
each of p and q, independently, is 0 or 1;
each of r and t, independently, is 1, 2, or 3; and
each of u and v, independently, is 0, 1, 2, 3, 4, 5, 6, 7, or 8.

2. The method of claim 1, wherein the anti-HCV compound is of formula (II):

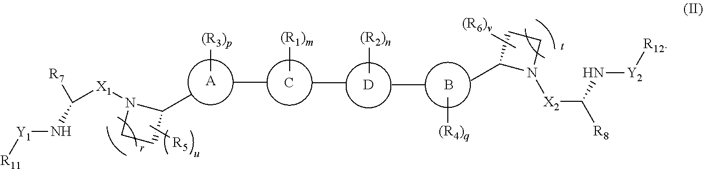

3. The method of claim 1, wherein the anti-HCV compound is of formula (III):

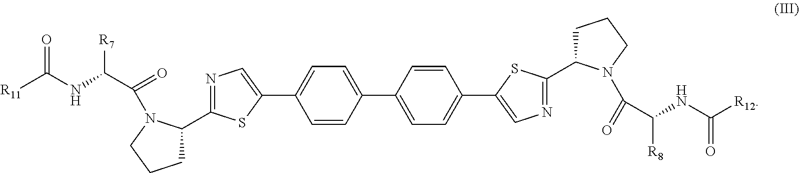

4. The method of claim 1, wherein the anti-HCV compound is:

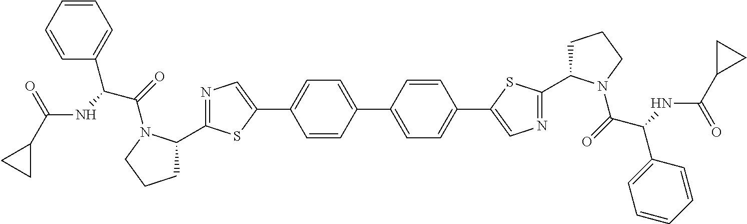 [SMILES: O=C(N[C@@H](C(=O)N1CCC[C@H]1c1ncc(-c2ccc(-c3ccc(-c4cnc([C@@H]5CCCN5C(=O)[C@H](NC(=O)C5CC5)c5ccccc5)s4)cc3)cc2)s1)c1ccccc1)C1CC1]

5. The method of claim 1, wherein the anti-HCV compound is:

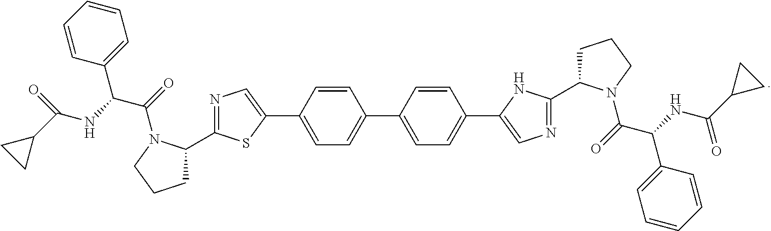 [SMILES: O=C(N[C@@H](C(=O)N1CCC[C@H]1c1ncc(-c2ccc(-c3ccc(-c4cnc([C@@H]5CCCN5C(=O)[C@H](NC(=O)C5CC5)c5ccccc5)s4)cc3)cc2)[nH]1)c1ccccc1)C1CC1]

6. The method of claim 4, wherein an HCV NS3 inhibitor is administered.

7. The method of claim 6, wherein the HCV NS3 inhibitor is telaprevir.

8. The method of claim 6, wherein the HCV NS3 inhibitor is boceprevir.

9. The method of claim 4, wherein an HCV NS5B inhibitor is administered.

10. The method of claim 9, wherein the HCV NS5B inhibitor is sofosbuvir.

11. The method of claim 4, wherein the HCV inhibitor is ribavirin.

12. The method of claim 4, wherein an IFN-α is administered.

13. The method of claim 12, wherein the IFN-α is a pegylated-IFN-α.

14. The method of claim 4, wherein two HCV inhibitors of (a) are administered.

15. The method of claim 5, wherein an HCV NS3 inhibitor is administered.

16. The method of claim 15, wherein the HCV NS3 inhibitor is telaprevir.

17. The method of claim 15, wherein the HCV NS3 inhibitor is boceprevir.

18. The method of claim 5, wherein an HCV NS5B inhibitor is administered.

19. The method of claim 18, wherein the HCV NS5B inhibitor is sofosbuvir.

20. The method of claim 5, wherein the HCV inhibitor is ribavirin.

21. The method of claim 5, wherein an IFN-α is administered.

22. The method of claim 21, wherein the IFN-α is a pegylated-IFN-α.

23. The method of claim 5, wherein two HCV inhibitors of (a) are administered.
